(19) United States
(12) Patent Application Publication
(10) Pub. No.: US 2012/0189560 A1
(43) Pub. Date: Jul. 26, 2012

(54) Sugar-free Beverage Composition

(75) Inventor: Ammar A. Idlibi, Bristol, CT (US)

(73) Assignee: Healthy Sweet Beverages, LLC.

(21) Appl. No.: 13/012,792

(22) Filed: Jan. 24, 2011

(51) Int. Cl. A61K 8/34; A23L 2/60; A61Q 11/00; A23L 2/66; A23L 2/52; A23L 2/38; A23L 2/56; A23L 2/58

(52) U.S. Cl. 424/52; 426/590; 426/72; 426/73; 426/74; 426/66; 426/2

(57) ABSTRACT

A beverage composition for strengthening and protecting tooth structure from decay and cavities, and for promoting dental health is provided. The beverage composition comprises one or more of about 0.5% to about 5% by weight of a polyol, about 0.005% to about 25% by weight of a natural and/or an artificial sweetener, about 1% to about 30% by weight of one or more vitamins and minerals, and their derivatives, a soluble source of fluoride ions containing about 0.1 ppm to about 1 ppm of fluoride ions, about 0.002% to about 0.008% of a water soluble casein, predetermined quantities of an herbal and/or fruit extract, a licorice root extract obtained, for example, from Glycyrrhiza uralensis, a non-mineral nutritional supplement, a flavoring agent, a preservative, a coloring agent, and a sufficient amount of a base composition, for example, water. The pH of the beverage composition is about 3.5 to about 7.0.

### BACKGROUND

Dental caries, also known as tooth decay, is caused by oral bacteria. Oral bacteria damage hard tissues, for example, enamel, dentin, and cementum, of tooth structure, thereby producing dental caries. Organic acids produced by a process of fermentation of dietary sugar and the presence of dental plaque produced by odontopathogenic bacteria, demineralize the hard tissues of the tooth structure and thereby initiate dental caries. Sweetened and carbonated beverages contain sugars, for example, sucrose, high-fructose corn syrup, or a form of fructose that occurs as a natural fruit sugar, etc. The oral bacteria ferment these sugars, thereby forming a base for dental caries.

Tooth enamel is primarily weakened by frequent consumption of acidic beverages and by the acidity of plaque that is not brushed regularly and effectively. Consuming food products containing sugar such as sucrose weakens the tooth enamel as sugar can be fermented by the oral bacteria, resulting in harmful acids. The mineral content of the tooth structure is sensitive to an increase in acidity or exposure to an acidic environment. Specifically, the tooth structure undergoes continuous demineralization and remineralization between the tooth structure and surrounding saliva. When the pH at the surface of the tooth structure drops below 5.5, demineralization proceeds faster than remineralization, which further contributes to weakening of the tooth structure because of gradual loss of minerals from the tooth enamel. This makes the tooth structure more vulnerable to acid attacks from fermented sugars. Dental caries is aggravated by an increase in frequency of consumption of sugar containing food products and sweetened beverages. The frequency of consumption, rather than the quantity of a sweetened beverage consumed is a critical factor in weakening the tooth structure by demineralization. According to the American Academy of General Dentistry, any beverage with a pH below 4.0 has a harmful potential effect on the tooth enamel especially when sweetened with sugar and particularly if consumed in high frequency.

Children, for example, toddlers are particularly more vulnerable to dental caries as parents tend to feed them with sugar containing food products and beverages such as fruit juices, milk-containing by-products of fructose, maltose, etc. Since toddlers initially depend on a liquid diet, they are likely to consume milk containing products and by-products frequently on a regular basis. The frequent consumption of beverages containing natural sugar is a leading cause of dental caries in children. Often, pediatric dentists admit children and toddlers to the hospital for oral rehabilitation and dental surgeries for treating dental caries.

Parents are often deceived by marketing strategies of companies selling sweetened beverages, where the companies use advertising gimmicks and claims, for example, “no sugar added”, “100% natural juice”, etc. These advertising strategies and flashy claims mislead parents and allow the children to frequently consume sweetened beverages. Parents do not realize that the sweetened beverages contain natural sugar, for example, fructose, sucrose, etc., that ferments in the presence of oral bacteria and hence causes dental caries. The outcome is seen only when the children are diagnosed with caries, lesions or rampant caries, etc., as a result of frequent consumption of sweetened beverages. Some companies dilute the beverages with water and advertise that their food products contain 30% or 40% less sugar by volume of the product. However, even diluted beverages contain extracts of sugar, which ferments in the presence of oral bacteria and causes dental caries.

Hence, there is a long felt but unresolved need for a sugar-free beverage composition that strengthens and protects tooth structure from tooth decay and cavities, and that promotes dental health.

### SUMMARY OF THE INVENTION

This summary is provided to introduce a selection of concepts in a simplified form that are further described in the detailed description of the invention. This summary is not intended to identify key or essential inventive concepts of the claimed subject matter, nor is it intended for determining the scope of the claimed subject matter.

The sugar-free beverage composition disclosed herein addresses the above mentioned need for strengthening and protecting tooth structure from tooth decay and cavities, and for promoting dental health. The sugar-free beverage composition disclosed herein is free from natural sugar, but is sweetened with a sugar free natural ingredient. In an embodiment, the sugar-free beverage composition comprises a minimal percentage by weight of artificial sweeteners, ensuring safe consumption for children and others. The sugar-free beverage composition disclosed herein protects tooth enamel and prevents erosion of tooth structure. The sugar-free beverage composition disclosed herein further prevents demineralization of hard tissues on the surface of the tooth structure and also aids in strengthening weakened teeth.

The sugar-free beverage composition disclosed herein comprises about 0.5% to about 5% by weight of a polyol, about 0.005% to about 25% by weight of a sweetener, and a sufficient amount of a base composition, for example, purified water, to adjust the total weight percentage of the beverage composition to 100%. The polyol is selected from a group comprising, for example, xylitol, erythritol, maltitol, isomalt, lactitol, mannitol, sorbitol, hydrogenated starch hydrolysates, and any combination thereof. The sweetener is a natural sweetener, for example, stevia extracted from leaves of stevia rebaudiana, any other natural sweetener, or a combination thereof. The beverage composition has a pH in a range of about 3.5 to about 7.0. For example, the pH of the beverage composition is 4.1.

The sugar-free beverage composition disclosed herein further comprises about 1% to about 30% by weight of one or more vitamins and derivatives thereof. One or more vitamins are selected from a group comprising, for example, vitamin B12, vitamin B6, vitamin B3, vitamin B5, vitamin C, vitamin D3, and any combination thereof. The sugar-free beverage composition disclosed herein further comprises about 1% to about 8% by weight of one or more minerals and derivatives thereof. One or more minerals are selected from a group comprising, for example, calcium, phosphorous, and a combination thereof.

In an embodiment, the sugar-free beverage composition disclosed herein further comprises about 0.002% to about 0.008% of water soluble casein and one or more derivatives thereof comprising, for example, casein phosphopeptide-amorphous calcium phosphate. For example, the sugar-free beverage composition disclosed herein comprises about 0.002% to about 0.008% by weight of calcium caseinate. In an embodiment, the sugar-free beverage composition disclosed herein further comprises about 1 microgram to about 500 micrograms of an herbal extract per serving of about 6 ounces (oz) to about 8 oz of the beverage composition, about 0.01% by weight to about 40% by weight of a fruit extract, or a combination thereof. The beverage composition further comprises about 1 microgram to 200 micrograms of a licorice root extract obtained, for example, from Glycyrrhiza uralensis.

In other embodiments, the sugar-free beverage composition disclosed herein further comprises one or more of a recommended daily value (RDV) of about 5% to about 20% by weight of a non-mineral nutritional supplement, about 0.01% to about 2.0% by weight of a flavoring agent, and about 0.001% to about 0.05% by weight of a preservative. The non-mineral nutritional supplement is selected from a group comprising, for example, vitamin A, vitamin D, tocopherol, ascorbic acid, thiamine, riboflavin, vitamin B6, vitamin B12, vitamin K, vitamin B3, folic acid, biotin, and any combination thereof. The flavoring agent is selected from a group comprising, for example, a fruit flavoring agent extracted from, for example, apple, pear, pineapple, peach, lemon, lime, orange, apricot, grapefruit, tangerine, rhubarb, cassis, quince, passion fruit, papaya, mango, guava, litchi, kiwi, mandarin, coconut, and/or banana, a botanical flavoring agent, and any combination thereof. The preservative is selected from a group comprising, for example, benzoates such as sodium benzoate, potassium benzoate, calcium benzoate, sodium sorbate, potassium sorbate, calcium sorbate, sodium citrate, potassium citrate, polyphophates such as sodium hexametaphosphate, antioxidants such as ascorbic acid, ethylenediaminetetraacetic acid, beta hydroxy acid, butylhydroxytoluene, tertiary butylhydroquinone, dehydroacetic acid, dimethyldicarbonate, ethoxyquin, heptylparaben, and any combination thereof.

In an embodiment, the sugar-free beverage composition disclosed herein comprises a coloring agent in the range of, for example, about 0.0001% to about 0.1% by weight of the beverage composition. In an embodiment, the sugar-free beverage composition disclosed herein further comprises caffeine in the range of, for example, about 0.001% to about 0.02% by weight of the beverage composition. The caffeine can be, for example, natural or synthetic in origin. In an embodiment, the sugar-free beverage composition disclosed herein further comprises an emulsifier in the range of, for example, about 3% to about 30% by weight of the beverage composition.

The sugar-free beverage composition disclosed herein further comprises one or more organic compounds and inorganic compounds of fluorine. The sugar-free beverage composition disclosed herein further comprises about 0.1 part per million to about 1 part per million of a soluble source of fluoride ions. The fluoride ions are selected from a group comprising, for example, sodium fluoride, calcium fluoride, fluorosilicic acid, fluorine, derivatives of fluorides, and any combination thereof. The soluble source of fluoride ions contains about 0.1 part per million to about 1 part per million of the fluoride ions. The soluble source of the fluoride ions is, for example, a soluble fluoride salt.

Disclosed herein is also a use of the beverage composition for administering a fluoride regimen to a subject in need thereof, where the beverage composition comprises about 0.5% to about 5% by weight of a polyol such as xylitol, erythritol, maltitol, etc., about 0.005% to about 25% by weight of a sweetener such as stevia or another natural sweetener and/or an artificial sweetener, about 0.1 part per million to about 1 part per million of a source of fluoride ions, and a sufficient amount of a base composition to adjust the total weight percentage of the beverage composition to 100%. Also, disclosed herein is a use of the beverage composition for enabling development of a taste for low sweetened food by continued administration to a subject.

Also, disclosed herein is a method for strengthening and protecting tooth structure from decay and cavities by administering to a subject in need thereof, the beverage composition disclosed herein. In an embodiment, the beverage composition is carbonated, for example, by adding about 0.5% to about 7% by volume of carbon dioxide to the beverage composition. The carbonation can be natural or synthetic. The beverage composition is also pasteurized, for example, by hot-filling pasteurization, aseptic packaging, ozonation, radiation, ultra-violet light radiation, high pressure pasteurization, membrane permeation, pulsed electric field pasteurization, sonication, and any combination thereof. The beverage composition is preserved using preservation techniques, for example, aseptic packing and/or heat treatment or thermal processing steps, for example, hot filling and tunnel pasteurization. These steps can be used to reduce, for example, yeast, mold and microbial growth in the beverage.

The beverage composition disclosed herein comprising tooth-friendly ingredients provides a healthy alternative to high-sugar beverages and conventional fruit juices that contribute to tooth decay and childhood obesity.

### DETAILED DESCRIPTION OF THE INVENTION

Disclosed herein is a sugar-free beverage composition for strengthening and protecting tooth structure from tooth decay and cavities, and for promoting dental health. The beverage composition disclosed herein prevents tooth decay and allows remineralization of weakened tooth structure with the additional benefits of vitamins, fluoride, soluble calcium, and/or soluble phosphorus. The beverage composition disclosed herein is sweetened with a combination of sugar alcohols known as polyols. The beverage composition disclosed herein aids in preventing tooth decay and provides a favorable oral environment for strengthening teeth that have been weakened by the acidity of other beverages and/or oral bacteria to increase their resistance to such acidity.

The beverage composition disclosed herein comprises about 0.5% to about 5% by weight of a polyol, about 0.005% to about 25% by weight of a sweetener, and a sufficient amount of a base composition, for example, purified water, to adjust the total weight percentage of the beverage composition to 100%.

The polyol is selected from a group comprising, for example, xylitol, erythritol, maltitol, isomalt, lactitol, mannitol, sorbitol, hydrogenated starch hydrolysates, and any combination thereof.

Xylitol is a naturally occurring sugar alcohol that delivers sweetness similar to sucrose, commonly known as table sugar, with 33% lesser calories. Xylitol exhibits antibacterial characteristics, which reduce levels of bacteria in plaque and saliva that contribute to tooth decay. Controlled studies demonstrate a reduction in tooth decay of about 30% to about 60% in groups using xylitol as a sugar substitute. Xylitol is a “tooth-friendly” non-fermentable sugar alcohol. A study on the efficacy of xylitol has indicated dental health benefits in tooth decay prevention.

Erythritol is a sugar alcohol that shares “tooth-friendly” characteristics with xylitol. Erythritol combines with xylitol to prevent gastrointestinal side effects such as bloating, gas, etc. Erythritol is about 60% to about 70% as sweet as sucrose and has minimal calories. In addition to having low calories, erythritol does not affect blood sugar levels, does not cause tooth decay, and is absorbed by the body easily and is therefore unlikely to cause gastric side effects unlike other sugar alcohols.

Maltitol is a sugar alcohol and used as a low calorie sweetener. Maltitol does not metabolize in the presence of oral bacteria and therefore does not promote tooth decay. Maltitol has about 75% to about 90% of the sweetness of sucrose and has nearly identical chemical properties compared to sucrose, except for a color change, for example, browning, when heated. Maltitol is used as a replacement for sucrose since maltitol has lower calories, does not promote tooth decay, and has lower effect on blood sugar levels.

Isomalt is a sugar alcohol having sugar-like physical properties. Isomalt exhibits “tooth-friendly” characteristics like xylitol. Isomalt has a low impact on blood sugar levels and does not promote tooth decay. Lactitol is a sugar alcohol that is used as a low calorie sweetener. Lactitol has about 40% of the sweetness of sucrose. Lactitol is observed to exhibit “tooth friendly” characteristics and does not promote tooth decay. Lactitol also has a lower impact on blood sugar levels. Sorbitol is a sugar alcohol and is alternatively referred to as glucitol. Sorbitol is used as a low calorie sweetener exhibiting nutritive characteristics as sorbitol provides dietary energy.

Mannitol is a sugar alcohol exhibiting medical properties, for example, as an osmotic diuretic agent and a weak renal vasodilator. Additionally, mannitol has lower impact on blood sugar levels and also functions as a low calorie sweetener. Mannitol exhibits a higher heat of solution than other sugar alcohols and its low solubility reduces the cooling effect, for example, the properties exhibited by mint in candies and chewing gums. Hydrogenated starch hydrolysate is a mixture of multiple sugar alcohols, for example, sorbitol, maltitol, etc., and is used as a sugar substitute as the mixture provides about 40% to about 90% of the sweetness of sugar. Hydrogenated starch hydrolysate is similar to sorbitol and is used as a sweetener and a moisture retaining ingredient Like xylitol, hydrogenated starch hydrolysate is not readily fermented by oral bacteria and is used to formulate the beverage composition that does not promote tooth decay.

The sweetener of the beverage composition is a natural sweetener, an artificial sweetener, or a combination thereof. The natural sweetener is, for example, stevia extracted from leaves of stevia rebaudiana or any other natural sweetener. Stevia, also known as sweet leaf or sugar leaf, is a derivative of stevia rebaudiana classified in the botanical family Asteraceae. Stevia is used as a natural sweetener and can be used to replace sucrose. Stevia has zero calories, does not promote tooth decay, and has a low impact on blood sugar levels. The beverage composition disclosed herein comprises, for example, about 0.005% to about 0.015% by weight of stevia. The beverage composition disclosed herein further comprises about 0.1% to about 20% by weight of an artificial sweetener, for example, sucralose, acesulfame potassium, aspartame, neotame, saccharin, etc. In an embodiment, the beverage composition comprises a mixture of one or more of the polyols and stevia or another natural sweetener, and any combination thereof. In another embodiment, the beverage composition comprises a mixture of one or more of the polyols and an artificial sweetener and any combination thereof. In another embodiment, the beverage composition comprises a mixture of one or more of the polyols, stevia, an artificial sweetener, and any combination thereof.

The beverage composition disclosed herein further comprises about 1% to about 30% by weight of one or more vitamins and derivatives thereof. One or more vitamins are selected from a group comprising, for example, vitamin B12, vitamin B6, vitamin B3, vitamin B5, vitamin C, vitamin D3, and any combination thereof. Vitamin B12 maintains the health of nerve cells and red blood cells. Vitamin B12 is also vital for formation of deoxyribonucleic acid (DNA), which is the genetic material in cells. Vitamin B12 also metabolizes carbohydrates, proteins, and fats. Vitamin B6 is needed for more than 100 enzymes involved in protein metabolism. The central nervous system and immune systems need vitamin B6 to function efficiently. Vitamin B6 also facilitates conversion of tryptophan, which is an amino acid, to niacin. Vitamin B6 facilitates production of hemoglobin, which carries oxygen to tissues within the red blood cells. Vitamin B6 also aids in increasing the amount of oxygen carried by hemoglobin.

Vitamin B3, also known as niacin, is a complex form of vitamin B necessary for healthy skin, hair, eyes, and liver. Vitamin B3 also helps in the functioning of the nervous system and aids production of various stress related hormones in the adrenal glands and other parts of the human body. Vitamin B3 improves circulation and reduces cholesterol levels in the blood. Vitamin B5, also known as pantothenic acid, is needed to form coenzyme-A (CoA), and is critical in the metabolism and synthesis of carbohydrates, proteins, and fats. Vitamin B5 promotes anti-stress effects, energy metabolism, and detoxification. Vitamin C acts as an anti-oxidant, since vitamin C protects the human body against oxidative stress. Vitamin D3, also known as cholecalciferol, is naturally produced in skin when exposed to direct sunlight. Vitamin D3 is required for bone health and for preventing conditions such as depression, back pain, cancer, insulin resistance, pre-eclampsia during pregnancy, impaired immunity, and macular degeneration.

The beverage composition disclosed herein further comprises about 1% to about 8% by weight of one or more minerals and derivatives thereof. One or more minerals are selected from a group comprising, for example, calcium, phosphorous, and a combination thereof. According to the National Osteoporosis Foundation (NOF), calcium plays an important role in building stronger, denser bones early in life and in keeping bones strong and healthy later in life. Approximately 99% of the calcium in the body is stored in the teeth and bones. Next to calcium, phosphorous is the most abundant mineral in the body. Phosphorous and calcium work closely together to build strong bones and teeth. About 85% of the phosphorous in the body is found in the bones and teeth. Phosphorous is also present in the cells and tissues throughout the body. Phosphorous helps filter out waste in the kidneys and plays a role in how the body stores and uses energy.

The beverage composition disclosed herein further comprises one or more organic compounds and inorganic compounds of fluorine. For example, the beverage composition disclosed herein comprises about 0.1 part per million to about 1 part per million of a soluble source of fluoride ions. In another example, the beverage composition disclosed herein comprises about 0.025% to about 0.1% by weight of a source of fluoride ions. The soluble source of fluoride ions is, for example, a soluble fluoride salt. The fluoride ions are selected from a group comprising, for example, sodium fluoride, calcium fluoride, fluorosilicic acid, fluorine, derivatives of fluorides, and any combination thereof. The soluble source of fluoride ions contains, for example, about 0.1 part per million to about 1 part per million of the fluoride ions. Fluoride-containing compounds are used in topical and systemic fluoride therapy for preventing tooth decay. Disclosed herein is also a use of the beverage composition disclosed herein for administering a fluoride regimen to a subject in need thereof, where in addition to about 0.5% to about 5% by weight of a polyol, about 0.005% to about 25% by weight of a sweetener, the beverage composition comprises a soluble source of fluoride ions. For the prescription strength form of the beverage, the beverage composition comprises about 0.25 mg to about 1 mg of the fluoride ions obtained from premeasured containers. The beverage composition disclosed herein is therefore used as a vehicle to deliver daily prescribed fluoride dosing.

The beverage composition disclosed herein further comprises about 0.002% to about 0.008% of water soluble casein and one or more derivatives thereof. For example, the beverage composition comprises about 0.002% to about 0.008% by weight of calcium caseinate. One or more of the derivatives of the water soluble casein comprise, for example, casein phosphopeptide-amorphous calcium phosphate. Casein phosphopeptide-amorphous calcium phosphate (CPP-ACP) is known as recaldent. CPP-ACP is a milk derived product that strengthens and remineralizes the tooth structure and also helps in preventing tooth decay. Casein phosphopeptides (CPP) slows the process of tooth decay and demonstrates characteristics of remineralization of the tooth structure in early stages of tooth decay by making up for the lost calcium and phosphate ions due to tooth decay.

In an embodiment, the beverage composition disclosed herein further comprises about 1 microgram to about 500 micrograms of an herbal extract per serving of about 6 ounces to about 8 ounces of the beverage composition, about 0.01 to about 40% by weight of a fruit extract, or a combination thereof. A single serving of the beverage refers to an amount of, for example, about 6 ounces (oz) to about 8 oz of the beverage. The herbal extract is obtained from herbs selected from the group comprising, for example, astragalus, jujube date, coriolus versicolor, rhodiola rosea, Siraitia grosvenorii, Glycyrrhiza uralensis, and combinations thereof. For example, the beverage composition disclosed herein comprises about 1 microgram to about 200 micrograms of a licorice root extract obtained, for example, from Glycyrrhiza uralensis. Glycyrrhiza uralensis, a licorice root extract, has been particularly determined to reduce primary bacteria such as Streptococcus mutans responsible for tooth decay. In an embodiment, the beverage composition disclosed herein further comprises at least one additional extract of a plant belonging to, for example, Citrus species, Crataegus species, Panax species, Lavendula species, Melissa species, Avena species, and Tilia species.

The beverage composition disclosed herein further comprises one or more of a recommended daily value (RDV) of about 5% to about 20% by weight of a non-mineral nutritional supplement, about 0.01% to about 2% by weight of a flavoring agent, and about 0.001% to about 0.05% by weight of a preservative. The non-mineral nutritional supplement is selected from a group comprising, for example, vitamin A, vitamin D, tocopherol, ascorbic acid, thiamine, riboflavin, vitamin B6, vitamin B12, vitamin K, folic acid, vitamin B3, biotin, and any combination thereof. The flavoring agent is selected from a group comprising, for example, a fruit flavoring agent derived from, for example, apple, pear, pineapple, peach, lemon, lime, orange, apricot, grapefruit, tangerine, rhubarb, cassis, quince, passion fruit, papaya, mango, guava, litchi, kiwi, mandarin, coconut, and/or banana, a botanical flavoring agent, and a combination thereof. In an embodiment, the beverage composition disclosed herein is free from any flavoring agent. The preservative is selected from a group comprising, for example, sodium benzoate, potassium benzoate, calcium benzoate, sodium sorbate, potassium sorbate, calcium sorbate, sodium citrate, potassium citrate, sodium hexametaphosphate, ascorbic acid, ethylenediaminetetraacetic acid (EDTA), beta hydroxy acid (BHA), butylhydroxytoluene (BHT), tertiary butylhydroquinone (TBHQ), dehydroacetic acid, dimethyl dicarbonate, ethoxyquin, heptylparaben, and any combination thereof. The pH of the beverage composition is, for example, in a range of about 3.5 to about 7.0. For example, the pH of the beverage composition is 4.1.

In another embodiment, the beverage composition disclosed herein further comprises a coloring agent or dye. The amount of coloring agent used varies, for example, depending on the type of coloring agent used and the intensity desired in the finished beverage. The beverage composition disclosed herein comprises the coloring agent in the range of, for example, about 0.0001% to about 0.5% by weight of the beverage composition. In another embodiment, the beverage composition comprises the coloring agent in the range of, for example, about 0.001% to about 0.1% by weight of the beverage composition. In another embodiment, the beverage composition comprises the coloring agent in the range of, for example, about 0.004% to about 0.1% by weight of the beverage composition.

In other embodiments, the sugar-free beverage composition disclosed herein further comprises caffeine in the range of, for example, about 0.001% to about 0.02% by weight of the beverage composition. The caffeine is, for example, natural or synthetic in origin. In an embodiment, the sugar-free beverage composition disclosed herein further comprises an emulsifier in the range of, for example, about 3% to about 30% by weight of the beverage composition. In another embodiment, the beverage composition further comprises additional ingredients, for example, caramel, anti-foaming agents, gums, tea solids, cloud components, and mineral and non-mineral nutritional supplements.

The pH of the sugar-free beverage composition disclosed herein can be regulated by adding small amounts of alkaline agents. The alkaline agent is selected from a group comprising, for example, potassium hydroxide, sodium hydroxide, potassium carbonate, etc. The beverage composition comprises the alkaline agent, for example, potassium hydroxide, in the range of about 0.02% to about 0.04% by weight of the beverage composition. In an embodiment, typically about 0.03% of potassium hydroxide is used to regulate the pH to a desired optimum level. Additionally, preservatives are also used to regulate the pH of the beverage. The preservatives comprise, for example, derivatives of one or more preservatives and/or an optional dissolved preservative formulation. Beverage compositions with pH values of, for example, about 3 to about 4 are microstable and resist the growth of microorganisms in the beverage composition which makes the beverage composition suitable for consumption even after a long period of storage. The preservatives are selected from a group comprising, for example, benzoates such as sodium benzoate, potassium benzoate, calcium benzoate, etc., sorbates such as sodium sorbate, potassium sorbate, calcium sorbate, etc., citrates such as sodium citrate, potassium citrate, etc., polyphophates such as sodium hexametaphosphate, antioxidants such as ascorbic acid, ethylenediaminetetraacetic acid, beta hydroxy acid, butylhydroxytoluene, tertiary butylhydroquinone, dehydroacetic acid, dimethyldicarbonate, ethoxyquin, heptylparaben, and any combination thereof. The level of preservative used typically is adjusted according to the planned final pH of the beverage composition, as well as an evaluation of the microbiological spoilage potential of the particular beverage formulation. The maximum amount of the preservative typically employed is about 0.05% by weight of the beverage composition.

Disclosed herein is also a method for strengthening and protecting tooth structure from decay and cavities by administering to a subject in need thereof, the beverage composition disclosed herein. In the method disclosed herein, the beverage composition is carbonated, for example, by adding about 0.5% to about 7% by volume of carbon dioxide to the beverage composition. The carbonation can be natural or synthetic. The beverage composition is also pasteurized, for example, by hot-filling pasteurization, aseptic packaging, ozonation, radiation, ultra-violet light radiation, high pressure pasteurization, membrane permeation, pulsed electric field pasteurization, sonication, or any combination thereof.

In an embodiment, the beverage is preserved using one or more beverage preservation techniques, for example, aseptic packing and/or heat treatment or thermal processing steps, for example, hot filling and tunnel pasteurization. These techniques are used to reduce, for example, yeast, mold and microbial growth in the beverage. Heat treatment comprises, for example, hot fill methods typically using high temperatures, for example, about 190° F. for a short period of time, for example, about 10 seconds. Tunnel pasteurization typically uses lower temperatures for a longer period of time, for example, about 160° F. for about 10 minutes to about 15 minutes. Retort pasteurization typically uses higher temperatures, for example, 250° F. for about 3 minutes to about 5 minutes at an elevated pressure, for example, at pressure above 1 atmosphere.

In an embodiment, the beverage composition disclosed herein is used for enabling development of a taste for low sweetened food by continued administration to a subject, where the beverage composition comprises about 0.5% to about 5% by weight of a polyol, about 0.005% to about 25% by weight of a sweetener, and a sufficient amount of a base composition to adjust the total weight percentage of the beverage composition to 100%. Young children and toddlers can be trained to develop a taste for lower sweet food products by introducing them to frequent sipping of drinks made from the beverage composition disclosed herein at an early age.

The sugar-free beverage composition disclosed herein may further comprise juices in the form of, for example, a concentrate, a puree, a single-strength juice and may include a single-strength fruit, berry, or vegetable juice, concentrates, purees, milks, or any combination thereof. In an embodiment, fruit based concentrates or juices are extracted from sources comprising, for example, plum, prune, date, currant, fig, grape, raisin, cranberry, pineapple, peach, banana, apple, pear, guava, apricot, saskatoon berry, blueberry, plains berry, prairie berry, mulberry, elderberry, barbados cherry, that is, acerola cherry, choke cherry, date, coconut, olive, raspberry, strawberry, huckleberry, loganberry, currant, dewberry, boysenberry, kiwi, cherry, blackberry, quince, buckthorn, passion fruit, sloe, rowan, gooseberry, pomegranate, persimmon, mango, rhubarb, papaya, litchi, lemon, orange, lime, tangerine, mandarin, grapefruit etc, or any combination thereof. The concentrate is used at a level of, for example, about 0.2% to about 40% by weight of the beverage composition. In another embodiment, the concentrate may be used at a level of, for example, about 1% to about 20% by weight of the beverage composition. The fruit based juices can be included in the beverage composition to adjust the flavor and/or increase the juice content of the beverage composition. Deflavored and decolored fruit juices may also be employed in the beverage composition.

Enumerated below are examples of the beverage composition that strengthens and protects tooth structure from decay and cavities, and that promotes dental health:

### Example 1

A beverage composition is prepared comprising about 3% by weight of erythritol, about 0.8% by weight of xylitol, about 1% by weight of maltitol, about 0.013% by weight of stevia, about 5% by weight of calcium, about 2% by weight of phosphorus, about 10% by weight of vitamin B3, about 10% by weight of vitamin B5, about 10% by weight of vitamin B6, about 10% by weight of vitamin B12, about 25% by weight of vitamin D3, about 0.005% by weight of calcium caseinate, with the balance of the beverage composition adjusted by a base composition, for example, purified water, to bring the total weight percentage of the beverage composition to 100%.

### Example 2

A beverage composition for administering a fluoride regimen to a subject in need thereof is prepared comprising about 3% by weight of erythritol, about 0.8% by weight of xylitol, about 1% by weight of maltitol, about 0.013% by weight of stevia, about 5% by weight of calcium, about 2% by weight of phosphorus, about 10% by weight of vitamin B3, about 10% by weight of vitamin B5, about 10% by weight of vitamin B6, about 10% by weight of vitamin B12, about 25% by weight of vitamin D3, about 0.005% by weight of calcium caseinate, and about 0.1 part per million to about 1 part per million of a soluble source of fluoride ions, with the balance of the beverage composition adjusted by a base composition, for example, purified water, to bring the total weight percentage of the beverage composition to 100%.

### Example 3

A beverage composition is prepared comprising about 3% by weight of erythritol, about 0.8% by weight of xylitol, about 1% by weight of maltitol, about 5% by weight of an artificial sweetener, about 5% by weight of calcium, about 2% by weight of phosphorus, about 10% by weight of vitamin B3, about 10% by weight of vitamin B5, about 10% by weight of vitamin B6, about 10% by weight of vitamin B12, about 25% by weight of vitamin D3, about 0.005% by weight of calcium caseinate, with the balance of the beverage composition adjusted by a base composition, for example, purified water, to bring the total weight percentage of the beverage composition to 100%.

### Example 4

A beverage composition is prepared comprising about 3% by weight of erythritol, about 0.8% by weight of xylitol, about 1% by weight of maltitol, about 0.013% by weight of stevia, about 5% by weight of an artificial sweetener, about 5% by weight of calcium, about 2% by weight of phosphorus, about 10% by weight of vitamin B3, about 10% by weight of vitamin B5, about 10% by weight of vitamin B6, about 10% by weight of vitamin B12, about 25% by weight of vitamin D3, about 0.005% by weight of calcium caseinate, with the balance of the beverage composition adjusted by a base composition, for example, purified water, to bring the total weight percentage of the beverage composition to 100%.

### Example 5

A beverage composition is prepared comprising about 3% by weight of erythritol, about 0.8% by weight of xylitol, about 1% by weight of maltitol, about 0.013% by weight of stevia, about 5% by weight of calcium, about 2% by weight of phosphorus, about 10% by weight of vitamin B3, about 10% by weight of vitamin B5, about 10% by weight of vitamin B6, about 10% by weight of vitamin B12, about 25% by weight of vitamin D3, about 0.005% by weight of calcium caseinate, about 1 microgram to 200 micrograms of licorice root extract obtained, for example, from Glycyrrhiza uralensis, with the balance of the beverage composition adjusted by a base composition, for example, purified water, to bring the total weight percentage of the beverage composition to 100%.

The foregoing examples have been provided merely for the purpose of explanation and are in no way to be construed as limiting of the present invention disclosed herein. While the invention has been described with reference to various embodiments, it is understood that the words, which have been used herein, are words of description and illustration, rather than words of limitation. Further, although the invention has been described herein with reference to particular means, materials and embodiments, the invention is not intended to be limited to the particulars disclosed herein; rather, the invention extends to all functionally equivalent structures, methods and uses, such as are within the scope of the appended claims. Those skilled in the art, having the benefit of the teachings of this specification, may affect numerous modifications thereto and changes may be made without departing from the scope and spirit of the invention in its aspects.

1. A beverage composition comprising:
about 0.5% to about 5% by weight of a polyol;
about 0.005% to about 25% by weight of a sweetener; and
a sufficient amount of a base composition to adjust total weight percentage of said beverage composition to 100%;
whereby said beverage composition is a sugar-free composition that strengthens and protects tooth structure from decay and cavities, and promotes dental health.

2. The beverage composition of claim 1, wherein said polyol is selected from a group comprising xylitol, erythritol, maltitol, isomalt, lactitol, mannitol, sorbitol, hydrogenated starch hydrolysates, and any combination thereof.

3. The beverage composition of claim 1, wherein said sweetener is stevia extracted from leaves of stevia rebaudiana.

4. The beverage composition of claim 1, wherein said sweetener is an artificial sweetener.

5. The beverage composition of claim 1, further comprising about 1% to about 30% by weight of one or more vitamins and derivatives thereof.

6. The beverage composition of claim 5, wherein said one or more vitamins are selected from a group comprising vitamin B12, vitamin B6, vitamin B3, vitamin B5, vitamin C, vitamin D3, and any combination thereof.

7. The beverage composition of claim 1, further comprising about 1% to about 8% by weight of one or more minerals and derivatives thereof.

8. The beverage composition of claim 7, wherein said one or more minerals are selected from a group comprising calcium, phosphorous, and a combination thereof.

9. The beverage composition of claim 1, further comprising one or more organic compounds and inorganic compounds of fluorine.

10. The beverage composition of claim 1, further comprising a soluble source of fluoride ions, wherein said fluoride ions are selected from a group comprising sodium fluoride, calcium fluoride, fluorosilicic acid, fluorine, derivatives of fluorides, and any combination thereof, wherein said soluble source of said fluoride ions contains about 0.1 part per million to about 1 part per million of said fluoride ions.

11. The beverage composition of claim 10, wherein said soluble source of said fluoride ions is a soluble fluoride salt.

12. The beverage composition of claim 1, further comprising about 0.002% to about 0.008% of a water soluble casein and one or more derivatives thereof.

13. The beverage composition of claim 12, wherein said one or more derivatives of said water soluble casein comprise casein phosphopeptide-amorphous calcium phosphate.

14. The beverage composition of claim 1, further comprising one of about 1 microgram to about 500 micrograms of an herbal extract in a serving of about 6 ounces to about 8 ounces of said beverage composition, about 0.01% to about 40% by weight of a fruit extract, and a combination thereof.

15. The beverage composition of claim 1, further comprising about 1 microgram to about 200 micrograms of a licorice root extract.

16. The beverage composition of claim 15, wherein said licorice root extract is obtained from Glycyrrhiza uralensis.

17. The beverage composition of claim 1, further comprising a recommended daily value of about 5% to about 20% by weight of a non-mineral nutritional supplement, wherein said non-mineral nutritional supplement is selected from a group comprising vitamin A, vitamin D, tocopherol, ascorbic acid, thiamine, riboflavin, vitamin B6, vitamin B12, vitamin K, vitamin B3, folic acid, biotin, and any combination thereof.

18. The beverage composition of claim 1, further comprising about 0.01% to about 2% by weight of a flavoring agent, wherein said flavoring agent is selected from a group comprising a fruit flavoring agent, a botanical flavoring agent, and any combination thereof.

19. The beverage composition of claim 1, further comprising about 0.001% to about 0.05% by weight of a preservative, wherein said preservative is selected from the group comprising sodium benzoate, potassium benzoate, calcium benzoate, sodium sorbate, potassium sorbate, calcium sorbate, sodium citrate, potassium citrate, sodium hexametaphosphate, ascorbic acid, ethylenediaminetetraacetic acid, beta hydroxy acid, butylhydroxytoluene, tertiary butylhydroquinone, dehydroacetic acid, dimethyldicarbonate, ethoxyquin, heptylparaben, and any combination thereof.

20. The beverage composition of claim 1, wherein pH of said beverage composition is in a range of about 3.5 to about 7.0.

21. The beverage composition of claim 1, further comprising one or more of about 0.0001% to about 0.5% by weight of a coloring agent, about 0.001% to about 0.02% of caffeine, and about 3% to about 30% by weight of an emulsifier.

22. The beverage composition of claim 1, wherein said base composition is water.

23. A beverage composition comprising:
about 2% to about 5% by weight of erythritol;
about 0.5% to about 1% by weight of xylitol;
about 0.5% to about 5% by weight of maltitol;
about 0.005% to about 0.015% by weight of stevia; and
a sufficient amount of a base composition to adjust total weight percentage of said beverage composition to 100%;
whereby said beverage composition is a sugar-free composition that strengthens and protects tooth structure from decay and cavities, and promotes dental health.

24. The beverage composition of claim 23, further comprising about 3% to about 8% by weight of calcium and about 1% to about 3% by weight of phosphorus.

25. The beverage composition of claim 23, further comprising about 8% to about 12% by weight of vitamin B3, about 8% to about 12% by weight of vitamin B5, about 8% to about 12% by weight of vitamin B6, about 8% to about 12% by weight of vitamin B12, about 8% to about 12% by weight of vitamin C, and about 22% to about 28% by weight of vitamin D3.

26. The beverage composition of claim 23, further comprising about 0.1% to about 20% by weight of an artificial sweetener.

27. The beverage composition of claim 23, further comprising about 0.002% to about 0.008% by weight of calcium caseinate.

28. The beverage composition of claim 23, further comprising a soluble source of fluoride ions, wherein said fluoride ions are selected from a group comprising sodium fluoride, calcium fluoride, fluorosilicic acid, fluorine, derivatives of fluorides, and any combination thereof, wherein said soluble source of said fluoride ions contains about 0.1 part per million to about 1 part per million of said fluoride ions.

29. The beverage composition of claim 23, further comprising one of about 1 microgram to about 500 micrograms of an herbal extract in a serving of about 6 ounces to about 8 ounces of said beverage composition, about 0.01% to about 40% by weight of a fruit extract, a recommended daily value of about 5% to about 20% by weight of non-mineral nutritional supplement, about 0.01% to about 2.0% by weight of a flavoring agent, and about 0.001% to about 0.05% by weight of a preservative.

30. A method for strengthening and protecting tooth structure from decay and cavities comprising:
preparing a beverage composition, comprising:
about 0.5% to about 5% by weight of a polyol, wherein said polyol is selected from a group consisting of xylitol, erythritol, maltitol, isomalt, lactitol, mannitol, sorbitol, hydrogenated starch hydrolysates, and any combination thereof;
about 0.005% to about 25% by weight of a sweetener, wherein said sweetener is stevia extracted from leaves of stevia rebaudiana;
about 0.01% to about 2% by weight of a flavoring agent, wherein said flavoring agent is selected from a group consisting of a fruit flavoring agent, a botanical flavoring agent, and any combination thereof;
about 0.0001% to about 0.5% by weight of a coloring agent;
a sufficient amount of a base composition to adjust total weight percentage of said beverage composition to 100%;
administering said beverage composition to a subject in need thereof.

31-32. (canceled)

33. The method of claim 30, wherein said beverage composition is carbonated by adding about 0.5% to about 7% by volume of carbon dioxide to said beverage composition.

34. The method of claim 30, wherein said beverage composition is pasteurized by one of hot-filling pasteurization, aseptic packaging, ozonation, radiation, ultra-violet light radiation, high pressure pasteurization, membrane permeation, pulsed electric field pasteurization, sonication, and any combination thereof.

35. A method for strengthening and protecting tooth structure from decay and cavities:
preparing a fluoride composition, comprising
about 0.5% to about 5% by weight of a polyol, wherein said polyol is selected from a group consisting of xylitol, erythritol, maltitol, isomalt, lactitol, mannitol, sorbitol, hydrogenated starch hydrolysates, and any combination thereof; about 0.005% to about 25% by weight of a sweetener, wherein said sweetener is stevia extracted from leaves of stevia rebaudiana; about 0.1 part per million to about 1 part per million of a source of fluoride ions; and a sufficient amount of a base composition to adjust total weight percentage of said beverage composition to 100%; and
administering said fluoride composition to a subject in need thereof.

36. The method of claim 35, wherein said fluoride ions are selected from a group consisting of sodium fluoride, calcium fluoride, fluorosilicic acid, fluorine, derivatives of fluorides, and any combination thereof, wherein said soluble source of said fluoride ions contains about 0.1 part per million to about 1 part per million of said fluoride ions.

37. A method for enabling development of a taste for low sweetened food by continued administration a beverage composition to a subject, comprising:
preparing a beverage composition, comprising about 0.5% to about 5% by weight of a polyol, wherein said polyol is selected from a group consisting of xylitol, erythritol, maltitol, isomalt, lactitol, mannitol, sorbitol, hydrogenated starch hydrolysates, and any combination thereof; about 0.005% to about 25% by weight of a sweetener, wherein said sweetener is stevia extracted from leaves of stevia rebaudiana; and a sufficient amount of a base composition to adjust total weight percentage of said beverage composition to 100%; and
administering said beverage composition to said subject.

38. The method of claim 30, further comprising adding about 0.002% to about 0.008% of a water soluble casein and one or more derivatives thereof to the beverage composition, wherein said one or more derivatives of said water soluble casein comprise casein phosphopeptide-amorphous calcium phosphate.

39. The method of claim 30, further comprising adding one of about 1 microgram to about 500 micrograms of an herbal extract in a serving of about 6 ounces to about 8 ounces of said beverage composition, about 0.01% to about 40% by weight of a fruit extract, and a combination thereof to the beverage composition.

40. The method of claim 30, further comprising adding about 1 microgram to about 200 micrograms of a licorice root extract to the beverage composition, wherein said licorice root extract is obtained from Glycyrrhiza uralensis.

41. The method of claim 30, further comprising adding a recommended daily value of about 5% to about 20% by weight of a non-mineral nutritional supplement to the beverage composition, wherein said non-mineral nutritional supplement is selected from a group consisting of vitamin A, vitamin D, tocopherol, ascorbic acid, thiamine, riboflavin, vitamin B6, vitamin B12, vitamin K, vitamin B3, folic acid, biotin, and any combination thereof.

42. The method of claim 30, further comprising adding about 0.001% to about 0.05% by weight of a preservative to the beverage composition, wherein said preservative is selected from the group consisting of sodium benzoate, potassium benzoate, calcium benzoate, sodium sorbate, potassium sorbate, calcium sorbate, sodium citrate, potassium citrate, sodium hexametaphosphate, ascorbic acid, ethylenediaminetetraacetic acid, beta hydroxy acid, butylhydroxytoluene, tertiary butylhydroquinone, dehydroacetic acid, dimethyldicarbonate, ethoxyquin, heptylparaben, and any combination thereof.

43. A method for strengthening and protecting tooth structure from decay and cavities comprising:
preparing a fluoride composition, comprising:
about 0.5% to about 5% by weight of a polyol, wherein said polyol is selected from a group consisting of xylitol, maltitol, isomalt, lactitol, mannitol, sorbitol, hydrogenated starch hydrolysates, and any combination thereof; about 0.005% to about 25% by weight of a sweetener;
administering said fluoride composition to a subject in need thereof.

44. A method for strengthening and protecting tooth structure from decay and cavities comprising:
preparing a fluoride composition, comprising
about 0.5% to about 5% by weight of a polyol, wherein said polyol is selected from a group consisting of xylitol, maltitol, isomalt, lactitol, mannitol, sorbitol, hydrogenated starch hydrolysates, and any combination thereof; about 0.005% to about 25% by weight of a sweetener, wherein said sweetener is stevia extracted from leaves of stevia rebaudiana; about 0.1 part per million to about 1 part per million of a source of fluoride ions; and a sufficient amount of a base composition to adjust total weight percentage of said beverage composition to 100%; and
administering said fluoride regimen to a subject in need thereof.

45. A method for enabling development of a taste for low sweetened foods in a subject, comprising:
preparing a beverage composition comprising about 0.5% to about 5% by weight of a polyol, wherein said polyol is selected from a group consisting of xylitol, erythritol, maltitol, isomalt, lactitol, mannitol, sorbitol, hydrogenated starch hydrolysates, and any combination thereof; about 0.005% to about 25% by weight of a sweetener; and a sufficient amount of a base composition to adjust total weight percentage of said beverage composition to 100%; and
administering said beverage composition to said subject.
